(19) United States
(12) Patent Application Publication
(10) Pub. No.: US 2009/0246285 A1
(43) Pub. Date: Oct. 1, 2009

(54) MONODISPERSED ORGANIC MONOLAYER COATED CALCIUM-CONTAINING NANOPARTICLES

(76) Inventors: Francesco Stellacci, Somerville, MA (US); Oktay Uzun, Cambridge, MA (US); Sherman Cox, Allentown, PA (US)

Correspondence Address: Pearl Cohen Zedek Latzer, LLP, 1500 Broadway, 12th Floor, New York, NY, 10036, US

(21) Appl. No.: 12/396,700

(22) Filed: Mar. 3, 2009

Related U.S. Application Data
(60) Provisional application No. 61/064,386, filed on Mar. 3, 2008.

(51) Int. Cl. A61K 9/14; B05D 7/00; A61P 1/04; B05D 1/00; C08K 5/00; B32B 1/00; B32B 5/16; B32B 29/04; A23L 1/304; A23L 1/30

(52) U.S. Cl. 424/490; 427/215; 427/214; 427/180; 524/81; 428/403; 428/330; 426/289; 426/648; 977/773; 977/897; 977/915; 977/902; 977/961

(57) ABSTRACT

A method for the synthesis of monodispersed, organic-monolayer coated, calcium-containing nanoparticles is presented. A biphasic liquid system comprises an aqueous phase of bare particles and an organic phase containing organic molecules with carboxylic acid end group is mixed. The carboxylic acid group binds to the surface of the calcium-containing particles and the particles are coated with a monolayer of organic molecules. The exposed surface of the coated particles is more hydrophobic than the surface of the bare particle and the particles are extracted to the organic phase. The process changes the geometry of the particles and decreases the size distribution in a population of particles.

### CROSS-REFERENCE TO RELATED APPLICATIONS

This Application claims priority of U.S. Provisional Application Ser. No. 61/064,386, filed Mar. 3, 2008, which is hereby incorporated by reference, in its entirety.

### BACKGROUND OF THE INVENTION

The ability to control nanoparticle properties, such as composition, coating, size, uniformity, geometry and solubility, is of great importance for applications in material synthesis, composite materials, drug delivery, food and cosmetics. Solution based approaches have been utilized to date for the synthesis of nanoparticles from inorganic materials. The vast majority of these approaches, however, form multi-dispersed nanoparticles, i.e. a particle collection with a large size distribution. In addition, ways to manipulate the geometry of existing nanoparticles in a controlled way are limited. Moreover, solubility of nanoparticles in organic solvents remains a challenge, with synthesis involving nanoparticles being limited due to such low solubility.

An important type of nanoparticle is precipitated calcium carbonate (PCC) nanoparticles, which find use, inter alia, in the paper industry. Enhancing the physical, chemical and mechanical properties of PCC particles through low cost, high yield synthesis would, inter alia, improve paper characteristics such as brightness, opacity and bulk.

### SUMMARY OF THE INVENTION

In one embodiment, this invention provides a process for the preparation of monodispersed, organic-monolayer coated, calcium-containing nanoparticles, the process comprising:
forming a liquid biphasic system comprising an aqueous phase and an organic phase, wherein the aqueous phase comprises calcium-containing nanoparticles and the organic phase comprises a solution of at least one organic molecule containing at least one functional end group;
mixing the liquid biphasic system; and
collecting nanoparticles from the organic phase of the liquid biphasic system;
whereby the at least one organic molecule containing a functional end group interacts with the nanoparticles, such that the at least one organic molecule coats the nanoparticles.

In one embodiment the nanoparticles collected by the process are closely packed.

In one embodiment, the mixing is conducted at 80° c. for at least 12 hours. In another embodiment, the mixing is conducted under reflux.

In one embodiment, the functional end group is a carboxylic group.

In one embodiment, the collecting is accomplished by separating of the organic phase from the aqueous phase.

In one embodiment, the collecting is conducted by evaporation of the organic phase.

In one embodiment, the calcium-containing nanoparticle is a calcium carbonate nanoparticle.

In one embodiment, the nanoparticles have a diameter ranging from between 1 nm-100 nm. In one embodiment, the geometry of the nanoparticles collected as a product of the process are more spherical in shape than the nanoparticles introduced at the beginning of the process. In one embodiment, the size distribution of the nanoparticles collected as a product of the process is decreased in comparison to the distribution of the nanoparticles introduced at the beginning of the process. In one embodiment, the size distribution of the nanoparticles collected as a product of the process ranges between 5%-20% of the mean particle size.

In one embodiment, the particles form an ordered array, in which the nanoparticles are arranged as a single ordered layer, or in some embodiments, a multilayered ordered structure.

In one embodiment, the coating molecules comprise two or more organic molecules. In one embodiment, the two or more molecules comprise at least one hydrophilic molecule and at least one hydrophobic molecule. In one embodiment, the molecular coating comprises 9-decenoic acid molecules and 4-phenyl butyric acid molecules.

In one embodiment, the nanoparticles collected as a product of the process have a coating comprising between 60%-98% of their surface area. In one embodiment, the nanoparticles collected as a product of the process have a coating comprising between 10%-40% of their surface area.

In one embodiment, at least one molecule comprises a carboxylic acid molecule containing an additional functional group. In one embodiment, the molecule comprises carboxylic acid molecules containing an additional amine functional group. In one embodiment, the coating has high density. In one embodiment, the coating has low density.

In one embodiment, the nanoparticles of this invention are thermally stable. In one embodiment, the nanoparticles of this invention are nontoxic for human consumption. In one embodiment the nanoparticles of this invention are administered as an antacid.

In one embodiment, a material of interest may be adsorbed or deposited on the organic monolayer coating. In some embodiments, such material of interest may comprise a metal, semiconductor or second organic molecules, for example, a molecule incorporated for its biological activity, a targeting moiety, a labeling moiety, and others, as will be appreciated by one skilled in the art.

In one embodiment, a metal coating is applied to the particles such that a metal ion precursor is introduced to an organic phase comprising the coated nanoparticles, and solution conditions are such that the exposed functional group of the organic layer promotes precipitation of a metal onto the calcium-containing nanoparticles.

In one embodiment, the application of a second or additional organic molecule as a coating to the particles comprises introducing a functionalized organic molecule to an organic phase comprising the nanoparticles, under conditions such that intermolecular forces between the second or additional organic molecules and particle surface promote bonding or adhesion of the second or additional organic molecule to the calcium-containing nanoparticles. In one embodiment, the processes as described herein may further comprise precipitation of the nanoparticles to form ordered arrays. In some embodiments, the ordered array may comprise a single ordered layer of the nanoparticles, or in some embodiments, a nanoparticles lattice comprising multiple ordered layers of the nanoparticles, which in some embodiments entails a lattice having a defined unit cell.

In some embodiments, the processes of this invention further comprise preparation of material coated with or comprising the nanoparticles as herein described. For example, and in some embodiments, the invention provides for a paper product comprising the nanoparticles of this invention, for example, when the nanoparticles comprise a coating for the paper product. In another embodiment the nanoparticles are used as a filler material. In one embodiment, the filler material is utilized in the preparation of a food or pharmaceutical product. In one embodiment, the filler material is utilized to enhance the mechanical or optical properties of a polymer or a polymer composite.

In one embodiment, this invention provides a device, apparatus or apparel comprising the nanoparticles of this invention/prepared according to a process of this invention. In one embodiment a paper or a drug is made, comprising the nanoparticles of this invention/prepared according to a process of this invention.

In one embodiment this invention provides a process for synthesizing a complex nanoparticle-organic structure, the process comprising:
(a) forming a liquid biphasic system comprising an aqueous phase and an organic phase, wherein the aqueous phase comprises calcium-containing nanoparticles and the organic phase comprises a solution of at least one organic molecule containing at least one functional end group;
(b) mixing the liquid biphasic system to form organic molecule coated calcium-containing nanoparticles; and
(c) linking a first organic molecule coating a nanoparticle with a second organic molecule coating another nanoparticle;
whereby the linking in step (c) results in the formation of an organic molecule-coated nanoparticle complex.

In one embodiment spacing between calcium-containing particles in the complex structure are from 1 nm-100 nm. In one embodiment, the complex structure is water permeable. In one embodiment, the complex structure serves as a filter or a membrane. In another embodiment, the complex structure serves as a coating or a filler material. In one embodiment, the complex structure serves as a column. In one embodiment, the complex structure serves as an antacid.

In one embodiment this invention provides highly monodispersed, organic-monolayer coated, calcium-containing nanoparticles. In one embodiment, calcium-containing material is calcium carbonate. In one embodiment, nanoparticle geometry is spherical. In one embodiment the nanoparticles have a diameter ranging from between 1 nm-100 nm. In one embodiment, the nanoparticles have a diameter ranging from between 5 nm-10 nm. In one embodiment, the nanoparticles coating thickness ranges from between 0.5 nm-3.0 nm. In one embodiment, the nanoparticles are closely-packed. In one embodiment, the size distribution of a particle population represents a normal distribution.

### BRIEF DESCRIPTION OF THE DRAWINGS

FIG. 1 is a photograph showing the reaction mixture of 1:10 PCC to ligand mix (DA:PBA)=(1 to 1).

FIG. 2 is a transmission electron microscopy (TEM) micrograph of PCC particles that have a specific surface area of about 80 m²/g and are made according to example 2 of U.S. Pat. No. 5,643,631 the disclosure of which is incorporated herein by reference. Bar=20 nm.

FIG. 3 is a TEM micrograph of the collected organic phase from a 1:10 PCC to ligand mixture (DA:PBA)=(1 to 1). A sample taken from the toluene layer was evaluated by TEM, which yielded array formation of the monodispersed particles. Bar=20 nm.

FIG. 4 is a lower magnification TEM micrograph of array formation of the monodispersed particles obtained from the 1:10 PCC to ligand mix (DA:PBA)=(1 to 1) reaction. Size distribution analysis on these particles was performed. Left and right images represent before and after particle selection and analysis using “image J” program analysis for size distribution. Bar=100 nm.

FIG. 5 plots the size distribution of particles obtained from the 1:10 PCC to ligand mix (DA:PBA)=(1 to 1) reaction. The average particle diameter is 6.8±1.0 nm and the number of particles counted was 747. The minimal particle diameter is 3.5±1.0 nm and the maximal particle diameter is 10.6±1.0 nm.

### DETAILED DESCRIPTION OF THE PRESENT INVENTION

This invention is directed, in some embodiments, to preparations of highly monodispersed, organic monolayer coated particles, and processes for the production of the same. The particles find application in a multitude of fields and settings, and represent, in some embodiments, an important element in the development of new diagnostics/imaging techniques, therapeutics, such as photothermal cancer therapy, drug delivery systems, tissue engineering applications, and others. In other embodiments the particles of this invention find use in the paper industry as paper fillers or coaters, where, inter alia, particles enhance the opacity, brightness and bulk properties of the paper. In other embodiments the particles of this invention find application in photonics as photonic crystals where an ordered multilayered array of particles reflects a defined wavelength of light. In other embodiments the particles of this invention find application as optical markers where the dimensions of a core-shell particle with a dielectric core and a metallic shell provide distinct scattering properties and optically-induced heating properties. In other embodiments the particles of this invention find application as additives to polymer or polymer composites in order to enhance their mechanical and optical properties. The particles of this invention can find application in separation and purification techniques and as acid-neutralizers.

The ability to arrive at highly monodispersed coated calcium-containing particles has been elusive, to date, with the processes as described herein representing an unexpected, reproducible means to obtain such preparations, in some embodiments, with the ability to form complex arrays, with spherical particles, which find broad application.

Nanoparticle Preparations of the Invention In one embodiment this invention provides highly monodispersed, organic-monolayer coated, calcium-containing nanoparticles.

In one embodiment, the calcium-containing material is calcium carbonate. In one embodiment the calcium carbonate is a precipitated form of calcium carbonate (PCC). In one embodiment the calcium carbonate is formed by passing carbon dioxide into a solution of calcium hydroxide: the calcium carbonate precipitates out, and this grade of product is referred to as a precipitate (precipitated calcium carbonate, also referred to herein as “PCC”). In one embodiment precipitation occurs according to the following chemical reaction: Ca(OH)₂+CO₂→CaCO₃+H₂O. In one embodiment the calcium carbonate is synthetically made. In one embodiment the CaCO₃ is the calcium carbonate found naturally in the following minerals and rocks: Aragonite, Calcite, Vaterite or (μ-CaCO₃), Chalk, Limestone, Marble and Travertine. In one embodiment the source of the calcium-containing material is biological. In one embodiment the source of the calcium-containing material is a shell of a marine animal or an egg shell.

In one embodiment, the calcium-containing material is calcium oxide. In one embodiment the calcium-containing material is calcium hydroxide. In one embodiment the calcium-containing material is an organocalcium. In one embodiment the calcium-containing material is calcium lactate. In one embodiment the insoluble calcium-containing material is calcium phosphate, calcium oxalate, calcium pyrophosphate or other water insoluble calcium salts. In one embodiment the calcium-containing material is crystalline. In one embodiment, the calcium-containing material is amorphous. In one embodiment the calcium-containing material contains at least one crystalline domain and at least one amorphous domain.

In one embodiment, the nanoparticle is thermally stable. In one embodiment the nanoparticles remain stable during or after particle coating and uncoating processes take place.

In one embodiment, nanoparticle geometry is spherical. In one embodiment the nanoparticles have a diameter ranging from between 1 nm-100 nm. In one embodiment the nanoparticles are closely-packed.

According to this aspect of the invention, and in some embodiments, the term “monodispersed” refers to a relatively uniform particles size distribution, such that, in a given preparation, nanoparticle diameter will not vary or deviate significantly from the mean particle diameter. In some embodiments, deviation from the median size is minimal. In some embodiments, such term may be considered to be synonymous to the term “small particle size distribution”.

In one embodiment the term “monodispersed” refers to a preparation comprising nanoparticles with a percent average deviation of less than 20%, from the mean particle size in a given preparation. In one embodiment the mean size distribution is defined by the mean value obtained of particle diameters for a population of spherical particles. In one embodiment the mean diameter for a population of particles is about 6.5-7 nm, or in one embodiment, 6-7 nm, or in another embodiment, 6.7-6.9 nm, or in another embodiment, 6.8 nm. In one embodiment, the average deviation from the mean for such particles will be less than 1 nm. In some embodiments, the particle diameter of a population may range from about 3 nm to about 12 nm, or in another embodiment from about 1 nm to about 15 nm. As exemplified herein, one preparation obtained ranged in size from about 3.5 nm to about 10.6 nm. In one embodiment, the size distribution of the nanoparticles collected as a product of the process ranges between 5%-20% of the mean particle size. In one embodiment, the size distribution of the nanoparticles serving as a starting material for the process ranges between 40%-70% of the mean particle size. In one embodiment the size distribution of the nanoparticles collected as a product of the process is less variable in comparison to the size distribution of the nanoparticles serving as a starting material for the process.

In one embodiment the highly monodispersed properties of the nanoparticle preparation facilitate their arrangement in an ordered, closely packed array. In one embodiment particles can form closely packed arrays, which in turn are of a thickness comparable to the diameter of a particle, or “one-particle thick”. Such arrays may be referred in some embodiments, to being “essentially 2 dimensional”, in that the array thickness is comparable to the thickness of the dispersed particles. In one embodiment, the term “two-dimensional array” will refer to the arrangement of the monodispersed particles on a substrate, or as a solid layer, which is relatively uniform in terms of particle diameter.

In another embodiment, the highly monodispersed properties of the nanoparticle preparation facilitate their arrangement in an ordered, closely packed array in multiple layers, for example being several particle layers thick. Such arrays, may be considered to be “three dimensional arrays” in that the thickness of such arrays exceeds that of roughly the diameter of a single nanoparticle.

In one embodiment the three-dimensional arrays of this invention may comprise a lattice comprising multiple layers of particles, arranged in a uniform pattern, or in other embodiments, according to a desired pattern, which may be periodic, or vary with the dimensions of the array. In one embodiment the array of particles comprises a periodic arrangement having a periodicity that affects the motion of photons, for example, those having a wavelength of soft X-ray.

In one embodiment the average particle size is between 5 nm and 10 nm. In one embodiment average particle size is between 1 nm and 10 nm. In one embodiment the average particle size is between 0.1 nm and 1 nm. In one embodiment the average particle size is between 1 nm and 100 nm.

In one embodiment the average particle size is between 1 nm and 20 nm. In one embodiment the average particle size is between 10 nm and 20 nm.

In one embodiment the particles are spherical and in another embodiment the particles are non-spherical.

The nanoparticles of this invention comprise a coating which comprises an organic molecule with at least one functional end group. In one embodiment the functional end group is a carboxylic acid group. In one embodiment, the functional end group serves as the anchor of a rod-like organic molecule to the particle. Once an anchor is made between the functional end group and the nanoparticles, neighboring organic molecules that have similar rod-like structure are self-assembled with their long axis perpendicular to the particle surface. Two molecules are held stretched in this way due to the van der Waals forces between the long “tails” of the molecules. Such arrangement forms a packed mono-molecular layer on the surface of the particle. When the other end of the molecule is kept hydrophobic, the surface of the coated particle becomes hydrophobic as well. This hydrophobicity will become effective once a monolayer of functional molecules is self-assembled on the particles. This hydrophobicity renders the particle soluble in organic solvent and transfer the coated particle from the aqueous phase into the organic phase.

In one embodiment, the process results in nanoparticles having a coating comprising between 60%-98% of their surface area. In one embodiment, the process results in nanoparticles having a coating comprising between 10%-40% of their surface area.

In one embodiment, an additional functional group is present on the self-assembled molecules. The additional functional group is located at the molecule end that is exposed to the environment. In one embodiment this additional functional group is used for linking other molecules to the particle. In one embodiment this additional functional group is a carboxylic acid, an amine, a biotin, hydroxyl, ethylene glycol, an unsaturated hydrocarbon, or a phenyl. In some embodiments the functional group resembles the polar end group of natural and synthetic lipids. In some embodiments the additional functional groups can be bound to a protein or a DNA molecule. In one embodiment the additional exposed functional group can bind the nanoparticles to a cell receptor or to a cell membrane. In one embodiment the additional functional group form links between two nanoparticles. In one embodiment the additional functional group or the molecule bound to it represent a targeting moiety, for use in in vivo applications. In one embodiment the targeting moiety bound through the surface-exposed additional functional groups is used for tissue targeting. In one embodiment the targeting moiety binds to receptors on cells. In one embodiment the targeting moiety adheres to cell membranes. In one embodiment a cleavable moiety is bounded through the functional group. In one embodiment cleavable moiety is used for controlled drug release. In one embodiment the functional group or the molecule bound to it is a fluorescent marker. In one embodiment the functional group or the molecule bound to it are used for immunoassays.

In one embodiment a multilayer of bi-functional molecules is constructed on the particles. In one embodiment a multilayer structure of bifunctional molecules increases the size of the particle.

In one embodiment a multilayer of bifunctional molecules links two particles while keeping a larger distance between the particles. In one embodiment constructing multilayers of bifunctional molecules on particles and linking molecules of different particles creates a complex organic-nanoparticle structure. In one embodiment this structure has improved mechanical properties. In one embodiment this structure is porous. In one embodiment this structure is water permeable. In one embodiment, the term “water permeable” refers to pores in the structure creating a continuous pathway for water molecules to permeate throughout the structure. In one embodiment, water molecules entering one end of the structure can flow through the structure and exit through another end. In one embodiment, water molecules can carry a chemical or a mixture of chemicals or one or more solutes that will be transformed through the nanoparticles complex structure. In one embodiment this structure can be used as a filter or a membrane, where various chemicals have different affinity to the particle organic coating or the organic linkers, and at least one component of a solution is retained in the structure for a longer period than another chemical or solvent. In one embodiment instead of water, an organic solution can flow through the structure. In one embodiment the organic molecules coating the particles or linking between them are modified to retain at least one molecule of interest thus separating it from a mixture. In one embodiment the organic molecules are modified to become more hydrophilic. In one embodiment the organic molecules are modified to become more hydrophobic. In one embodiment the structure pores are modified to optimally retain gas phase molecules. In one embodiment the nanoparticles complex is further dissolved in an acid, thus enables the collection and purification of the retained molecule of interest that was trapped in the structure. In one embodiment the nanoparticles filter or membrane is part of a separation device. In one embodiment, in such device, at the end of the nanoparticles complex holder, a collection vial or tool is connected to collect the molecules and solvent that passed through the structure. In one embodiment collection is performed as a function of time to collect different species with different retention times. In one embodiment the nanoparticles separation filter and the collection module are parts in an automated system or a robot. In one embodiment an imaging or analyzing system is connected to the separation device comprising the nanoparticles of this invention.

In one embodiment the complex organic-nanoparticle structure of this invention may find application as a filler or a coating for paper, plastics, inorganic or metallic substrates. In one embodiment mixing the complex nanoparticles-organic structure with a paper, plastic or a metal substrate enhances the mechanical properties of the substrate.

In some embodiments, nanoparticles of this invention comprise a coating which comprises two or more functional end groups, or in some embodiments, two or more organic molecules. According to this aspect of the invention and in one embodiment, one of the organic molecules is a hydrophilic molecule and one organic molecule is a hydrophobic molecule. In one embodiment two or more organic molecules are hydrophobic. In one embodiment, the molecular coating comprises 9-decenoic acid molecules and 4-phenyl butyric acid molecules. In one embodiment at least one organic molecule contains a saturated or an unsaturated alkyl chain. In one embodiment at least one organic molecule contains an aromatic group. In one embodiment the molecule chain is straight. In one embodiment the molecule is branched. In one embodiment there are inter-molecular forces holding at least two organic molecules forming the particle coating. In one embodiment the intermolecular forces between coating molecules are van-der-Waals forces. In one embodiment the intermolecular forces are polar bonds. In one embodiment the intermolecular forces are hydrogen bonds. In one embodiment at least two molecules comprising the coating are covalently-bonded.

In some embodiments, the nanoparticle arrays and structures of this invention may comprise molecules of interest, incorporated within such arrays and structures, which are useful for application of the nanoparticles in various technologies. For example, and in some embodiments, the nanoparticle arrays and structures of this invention are modified to incorporate therapeutic compounds for biologic applications, for example, use of the nanoparticle arrays and structures of this invention may comprise acid-neutralization formulations for administration to subjects suffering from excess acidity, e.g. associated with ulcers. According to this aspect, other therapeutic compounds may be incorporated therein, for example, chemotherapeutics for H. pylori infection.

It is to be understood that any number of desirable compounds may be incorporated within the nanoparticle arrays and structures of this invention, and the chemistry to incorporate such molecules is well known, and is a function of the material being incorporated therein.

In one embodiment a composition comprising at least one organic molecule as part of a particle coating in the nanoparticle arrays and structures of this invention results in a decrease in the size distribution of the coated nanoparticles. In one embodiment, incorporation of at least one organic molecule in the particle coating influences the particle geometry. In one embodiment the selection of one or more organic molecules for the coating renders the particles more spherical in shape. In one embodiment the energetics of bond formation and bond breakage between the organic molecules, the nanoparticles and the solvent may induce changes in geometry and size of the particles. In one embodiment bond formation and bond breaking causes nanoparticle surface atoms rearrangement, resulting in a unique particle size or particle geometry or a combination thereof.

In one embodiment the majority of the coating molecules have a hydrophobic terminus that is surface exposed after monolayer formation. In one embodiment the majority of surface exposed hydrophobic groups, makes the particles soluble in organic solvent. In one embodiment solvation in organic solvent enables the extraction of the particles from the aqueous phase into the organic phase. In one embodiment a small percentage of the organic coating molecules have a hydrophilic end group exposed to the surface after monolayer formation. In one embodiment the hydrophilic molecules modify the total solubility of the particles. In one embodiment, some hydrophilic end groups exposed to the surface enables better solubility of the particles in less hydrophobic organic solvents. In one embodiment, the ratio of hydrophobic to hydrophilic exposed groups on the molecules forming the particle coating, fine-tunes the solubility of the molecules in a certain organic solvent.

In one embodiment a minority of hydrophilic-end molecules is utilized as precursors for linking additional molecules to the coating. In one embodiment isolated hydrophilic functional groups are used to bind a linker molecule that can link to particles. In one embodiment a number of hydrophilic functional groups on the surface of the coating are used to bind a biological marker, an antigen, a protein, a DNA, a fluorescent probe, or a drug.

In one embodiment covalent bonding between at least two organic molecules forming the coating occurs before or during the adsorption of the molecules to the particle. In one embodiment the covalent bonding between two coating molecules occurs after the molecules are attached to the particles. In one embodiment bonding between molecules is induced using chemicals in solution. In one embodiment bonding is induced using a light source or by other means of photochemistry. In one embodiment bonding is induced by physical changes. In one embodiment bonding is induced by changes of pH, temperature, solvent or concentrations of species in solution. In one embodiment bonding between coating molecules is induced by means of electrochemistry.

In one embodiment bonding between neighboring organic molecules coating the particle yields a polymer coating the particle. In one embodiment polymerization of organic molecules coating the particles is radical polymerization. In one embodiment ethene groups on adjacent organic molecules polymerize to give a polyethene layer surrounding the particle. In one embodiment polymerization of coating molecules makes the nanoparticle robust.

In one embodiment the particle surface is patterned by an organic molecule coating. In one embodiment at least two organic molecules coating the surface of the particle form domains on the particle surface. In one embodiment patterning can be controlled. In one embodiment controlled patterning depends on the curvature of the particle. In one embodiment the curvature of the spherical particle and the choice of the two molecules, forms ordered domains or rows of the at least two molecules on the surface. In one embodiment controlled patterning imparts special chemical properties to the particle. In one embodiment, the choice of the at least two molecules is such that when incorporated in a paper product, the product will have improved properties. In one embodiment improved properties of strength, brightness, opacity, gloss, or a combination thereof can be obtained.

In some embodiments the domains can be imaged or manipulated. In some embodiments the organic molecule domains can be imaged or manipulated using an AFM tip. In one embodiment, the coating has high density. In one embodiment, the coating has low density. In one embodiment coating comprises pores, making the calcium carbonate surface accessible to interaction with solvent, solution, or chemicals in solution or to gas molecules.

### I. Processes to Prepare the Nanoparticles and Nanoparticle Arrays of the Invention

In some embodiments, this invention provides for processes of preparation of the nanoparticles and arrays as described herein.

This invention provides, in some embodiments, a process for the preparation of monodispersed, organic-monolayer coated, calcium-containing nanoparticles, the process comprising:
forming a liquid biphasic system comprising an aqueous phase and an organic phase, wherein the aqueous phase comprises calcium-containing nanoparticles and the organic phase comprises a solution of a molecule containing a functional end group;
mixing the liquid biphasic system; and
collecting nanoparticles from the organic phase of the liquid biphasic system;

whereby the at least one organic molecule containing a functional end group interacts with the nanoparticles, such that the at least one organic molecule coats the nanoparticles.

In one embodiment, the term “liquid biphasic system” refers to a system comprising two immiscible liquid phases. In one embodiment the two phases are immiscible due to the energetics of the system. In one embodiment the system is more energetically stable when the two liquids are separated to form two phases. In one embodiment the energy of the system is in a lower, more stable state, when molecules in the same phase bond with each other as compared with a situation where molecules of one phase form bonds with molecules of another phase.

In one embodiment the separation of the two phases creates an interface between the two phases. In one embodiment, in this interface there are forces or bonds holding molecules of the two phases together. In one embodiment the interface between the two phases allows reaction between a molecule dissolved in one phase with a molecule or a particle dissolved or suspended in the other phase. In one embodiment, the product of this reaction can be soluble in one of the two phases. In one embodiment the product of the reaction taking place at the interface is soluble in the organic phase. In one embodiment the solubility of such product in one phase, enables the spontaneous transfer of the product of the reaction from the interface into the bulk of one of the phases. In one embodiment, interfacial reactions are highly controlled in terms of kinetics. In one embodiment interfacial reactions provides high control over product properties. In one embodiment interfacial reactions provides high control over product size and geometry. In one embodiment interfacial reactions provide high control over purity of the product and the ability to separate the product from at least one of the reactants.

In one embodiment once the reaction is complete the coated nanoparticles dissolve in the organic phase. In one embodiment the organic phase also contains free organic molecules not bound to the particles. In one embodiment, before particle collection from the organic phase, the free organic molecules are extracted from the organic phase using a basic aqueous solution. In one embodiment mixing the basic aqueous solution with the organic phase, results in the conversion of the organic acid molecules into organic salts that are soluble in the aqueous phase. In one embodiment the salt transfers to the aqueous phase. In one embodiment the organic phase is now cleaner, containing predominantly the coated nanoparticles.

In one embodiment the processes of this invention can be controlled such that a desirable distribution of nanoparticle size may be obtained. In one embodiment, such control of the processes of this invention, according to this aspect, may be a reflection of the choice of organic molecule used for particle coating and extraction.

In one embodiment, two or more organic molecules may comprise the coating of the nanoparticles as herein described. According to this aspect, and in one embodiment, such choice may influence the particle size, particle geometry and other array characteristics, for example, the geometry, and closely packed nature of the array.

In another embodiment, all aspects of the processes of this invention may influence array characteristics, or nanoparticle characteristics. For example, and in one embodiment, interaction between the functional end group of the organic molecule and the surface of the calcium-containing particle may influence the coating thickness, uniformity, overall size of the coated particle, etc., as well as array properties, including the closely-packed nature of the array, or other physical characteristics, such as wettability, stress, etc.

In another embodiment, the choice of organic solvent may influence array characteristics, or nanoparticle characteristics. In one embodiment, particle size is affected by the choice of solvent, or concentration of solutes in the solvent, or concentration of particles present, or concentration of organic molecule used, or a combination thereof.

In one embodiment the temperature, reaction time, solution pH or additives included in the reaction mixture may influence array characteristics, or nanoparticle characteristics, as will be appreciated by one skilled in the art.

It will be appreciated that verification of nanoparticle and/or array characteristics can be confirmed by any number of imaging or analysis techniques, known in the art, for example, and in one embodiment, by transmission electron microscopy (TEM), or in another embodiment by atomic force microscopy (AFM), or in another embodiment, by contact angle measurements, or others, as will be known to the skilled artisan.

In one embodiment, a process of this invention further comprises a step of removal of the organic monolayer from the particles. In one embodiment removal of the organic monolayer is performed for the purpose of coating the nanoparticles with a different material that adheres directly to the calcium-containing surface. In one embodiment this material modifies the nanoparticle properties such that their mechanical, electronic or optical properties are controlled. In one embodiment such nanoparticles characteristics find applications in the paper industry, in drugs or cosmetics products and in medical applications. According to this aspect of the invention, and in one embodiment, the preliminary organic molecule coating is initially used to decrease the size distribution of the particles or to give them a spherical shape, or to solvate the particles in the organic solvent. In some embodiments, once at least one of these goals is achieved, the organic coating monolayer can be removed. In one embodiment the organic layer is removed thermally. In one embodiment the organic layer is removed by oxidation. In one embodiment the organic layer is removed by oxidation in solution. In one embodiment the organic layer is removed after particles are dried. In one embodiment the organic layer is removed by etching the surface layer of the calcium-containing particle. In one embodiment the organic molecule is removed by chemical exchange. In one embodiment, after monolayer removal, the particles are coated with a metal. In one embodiment the metal-dielectric core-shell particles have distinct optical properties. In one embodiment the core shell particles serve for cancer diagnosis and therapy as described in detail herein below. In one embodiment the particles are coated with a metal or a semiconductor, for quantum dots or quantum computing applications. In one embodiment the particles are coated with a polymer or with a dye for paper filling and coating applications, and for electronic ink. In one embodiment the particles are coated with a porous polymer for the purpose of slow-release of calcium-containing material.

In one embodiment the organic monolayer coating or specific organic monolayer domains in the coating or specific organic molecules in the coating are chemically modified. In one embodiment the organic monolayer coating or molecules in it can be modified to contain an additional functional group. In one embodiment the additional functional group is an amine. In one embodiment the additional functional group is exposed to the surrounding. In one embodiment the additional functional group is used for further chemical synthesis. In one embodiment the additional synthesis involves linking an organic molecule to the molecule forming the coating. In one embodiment synthesis involves linking a metal ion to the organic molecule. In one embodiment synthesis comprises linking an inorganic compound, a metal atom, a metal cluster, or a polymer to the organic monolayer forming the particle coating. In one embodiment synthesis comprises linking a biological molecule or a biological function to the organic monolayer coating the particle

In one embodiment, additional material is physically adsorbed or deposited on top of the organic monolayer coating. In one embodiment, the additional material is a metal. In one embodiment the additional material is an inorganic compound. In one embodiment the additional material is an organic molecule. In one embodiment, the additional material is a polymer. In one embodiment encapsulation of the particle by a polymer is done in order to isolate it from its surroundings. In one embodiment encapsulation prevents aggregation of particles. In one embodiment encapsulation protects the particles against undesired chemical reactions. In one embodiment, encapsulation controls the rate at which calcium-containing material leaves the capsule. In one embodiment encapsulation by a polymer results in a monodispersed or spherical polymeric shell particles.

In one embodiment the polymeric material is polyethene or polypropylene, in one embodiment, the polymer is polystyrene. In one embodiment the polymer is a graft polymer, a copolymer or a polyelectrolyte. In one embodiment the polymer is branched. In one embodiment the polymer has high density and in another embodiment a low density. In one embodiment the polymer is a protein or a DNA or a polysaccharide. In one embodiment the polymer contains phenyl groups. In one embodiment the polymer contains amide groups. In one embodiment the polymer is a polyester or polyurethane. In one embodiment the polymer is biocompatible. In one embodiment the polymer is biodegradable. In one embodiment the polymer is transparent.

In another embodiment, the additional material adsorbed on or coating the nanoparticles is biological. In another embodiment, the additional material adsorbed on or coating the nanoparticles is a drug or therapeutic agent. In one embodiment the material is a protein. In one embodiment the material is a peptide. In one embodiment the material is a receptor. In one embodiment the material can bind to a receptor. In one embodiment the material is an antibody or an antigen. In one embodiment the material enables the bonding of a particle to a cell. In one embodiment bonding of particle to a cell induces drug release. In one embodiment the biological material slowly degrades or decomposes, gradually releasing the calcium-containing material for acid-neutralizing applications. In one embodiment the protein is fluorescent. In one embodiment, the biological material is a DNA, RNA, a nucleic acid or a nucleic acid sequence. In one embodiment the coated particle can pass through membrane channels. In one embodiment the additional material is adsorbed onto the particle from solution. In one embodiment adsorption involves covalent bonds. In one embodiment adsorption involves polar, ionic or van der Waals bonds. In one embodiment adsorption is reversible.

In one embodiment adsorption/desorption of material is controlled thermally. In one embodiment adsorption/desorption of material is controlled by pH change, chemical nature of the molecules involved, chemical environment and chemical concentration. In one embodiment the additional material is adsorbed from the gas phase. In one embodiment only part of the particle is coated by the adsorbed material. In one embodiment at least two types of additional materials are adsorbed on the particle.

It will be clear to a person skilled in the art that processes described herein, wherein a material is added to a particle of this invention, may be utilized for particles wherein the first organic monolayer remains attached to the particle surface, or for particles wherein the monolayer has been removed from the particles surface.

In one embodiment a process of this invention comprises preparing a liquid biphasic system using an aqueous phase and an organic phase. In one embodiment the aqueous phase comprises calcium-containing nanoparticles. In one embodiment the organic phase comprises a solution of at least one organic molecule containing at least one functional end group. In one embodiment the liquid biphasic system is mixed or stirred. In one embodiment the interaction of the organic molecules from the organic phase and the particles from the aqueous phase occurs at the interface between the organic and the aqueous phases. In one embodiment a higher interface area is obtained by mixing or stirring. In one embodiment higher interface area is obtained by heating. In one embodiment, the interaction between the organic molecules and the nanoparticles results in binding of the end group of the organic molecule to the particle. In one embodiment the “tail” of the molecule stretches away from the nanoparticles after the end group of the molecule is bound to the particle. In one embodiment the tail of the organic molecule is maintained in an extended configuration, with tails of neighboring molecules proximal thereto maintained in an extended configuration, as well. In one embodiment such stretched binding to a surface forms a mono-molecular layer of molecules on the nanoparticles. In one embodiment such monolayer formation is a spontaneous process. In one embodiment such monolayer formation is termed self-assembly.

In one embodiment, once a self-assembled organic monolayer have been formed, the mainly hydrophobic end groups of the organic molecules that are now exposed to the surface, makes the particles soluble in organic solvents. In one embodiment the coated nanoparticles are more hydrophobic than the non-coated nanoparticles. In one embodiment the coated nanoparticles are transferred spontaneously to the organic phase. In one embodiment the coated nanoparticles leave the aqueous phase. In one embodiment coated nanoparticles are collected from the organic phase of the liquid biphasic system.

In one embodiment the organic solvent of the biphasic system is toluene. In one embodiment the organic solvent is benzene, ether, hexane, or a mixture of two or more solvents. In one embodiment the organic solvent contains organic molecules dissolved in the solvent. In one embodiment the organic molecule comprises a carboxylic group. In one embodiment the organic molecule is Lauric (dodecanoic) acid. In one embodiment the organic molecule comprises a saturated or an unsaturated alkyl chain.

In one embodiment the organic molecule is rod-shaped. In one embodiment the organic molecule is branched. In one embodiment rod-shaped molecules enables a closed-packed organic monolayer to form around the particles. In one embodiment the rod like molecules has a length of between 0.6 nm and 2.2 nm. In one embodiment such length enables the best packing of the monolayer with fewer defects. In one embodiment such closed-pack array of rod-like molecules with exposed hydrophobic end-groups facilitates the transfer of the nanoparticles from the aqueous phase to the organic phase. In one embodiment branched molecules interfere with the packing of the monolayer. In one embodiment branched molecules can not make a high number of attractive intermolecular interactions with their neighboring molecules. In one embodiment having branched molecules in the monolayer results in more defects in the monolayer. In one embodiment such defects can make the nanoparticles permeable. In one embodiment such defects can result in contact between the calcium-containing material and materials in the environment of the particle.

In one embodiment the organic molecule represent the first generation of a dendrimer. In one embodiment dendrimer molecules on the particle can be the basis of a multiple generation dendrimeric structure. In one embodiment the dendrimeric structure is a vehicle for drug delivery. In one embodiment two or more organic molecules are dissolved in the solvent. In one embodiment two or more organic molecules contain a carboxylic group. In one embodiment one molecule is 9-decenoic acid and one molecule is 4-phenyl butyric acid molecules.

In one embodiment the organic phase contains 850 mg, 5 mmol of 9-decenoic acid and 820 mg and 5 mmol of 4-phenyl butyric acid dissolved in 10 mL of toluene. In one embodiment the concentration of the 9-decenoic acid is 0.5 M. In one embodiment the concentration of the 4-phenyl butyric acid is 0.5 M. In one embodiment the concentration of at least one of the organic acids is between 0.1 M and 0.5 M. In one embodiment the concentration of at least one organic acid is between 0.01 M and 0.1 M. In one embodiment the concentration of at least one organic acid is between 0.5 M and 1.0 M. In one embodiment the concentration ratio of two organic acids is between 1/1 and 1/10. In one embodiment the concentration ratio between two organic acids is between 1/10 and 1/100.

Example 1 describes one embodiment of a synthetic process for the preparation of monodispersed organic monolayer coated precipitated calcium-containing nanoparticles.

In one embodiment the aqueous phase comprises calcium-containing particles. In one embodiment the calcium-containing particles are calcium carbonate particles. In one embodiment the calcium carbonate particles are precipitated calcium carbonate particles (PCC particles). In one embodiment PCC particles have a specific surface area of about 80 m²/g and are made according to example 2 of U.S. Pat. No. 5,643,631, the disclosure of which is incorporated herein by reference. In one embodiment the solution comprises: 11.8% by weight aqueous PCC solution which contains 100 mg, 1 mmol of CaCO₃ nanoparticles diluted to 10 mL final volume with distilled water. In one embodiment final particle concentration is 0.1 M.

In one embodiment the liquid biphasic system comprises an organic phase and an aqueous phase. In one embodiment the organic phase comprises organic acid molecules in an organic solvent. In one embodiment the organic acid acts as a ligand for the particles. In one embodiment the concentration ratio of the ligand dissolved in the organic phase and the particles in the aqueous phase is 10/1. In one embodiment equal volumes of organic phase and aqueous phase are used. In one embodiment 10 mL of the organic phase is brought into contact with 10 mL of the aqueous phase. In one embodiment the total concentration of ligand in the organic phase is a sum of the concentrations of two ligands. In one embodiment total concentration of ligand is the sum of the concentrations of 9-decenoic acid and 4-phenyl butyric acid. In one embodiment the concentration of 9-decenoic acid is 0.5 M and the concentration of 4-phenyl butyric acid is 0.5 M, giving a total concentration of 1.0 M in the organic phase. In one embodiment, by mixing 10 mL aqueous phase comprising 0.1 M PCC particles with 10 mL organic phase comprising 1.0 M of ligand, the final mole ratio of ligand to particles is 10/1.

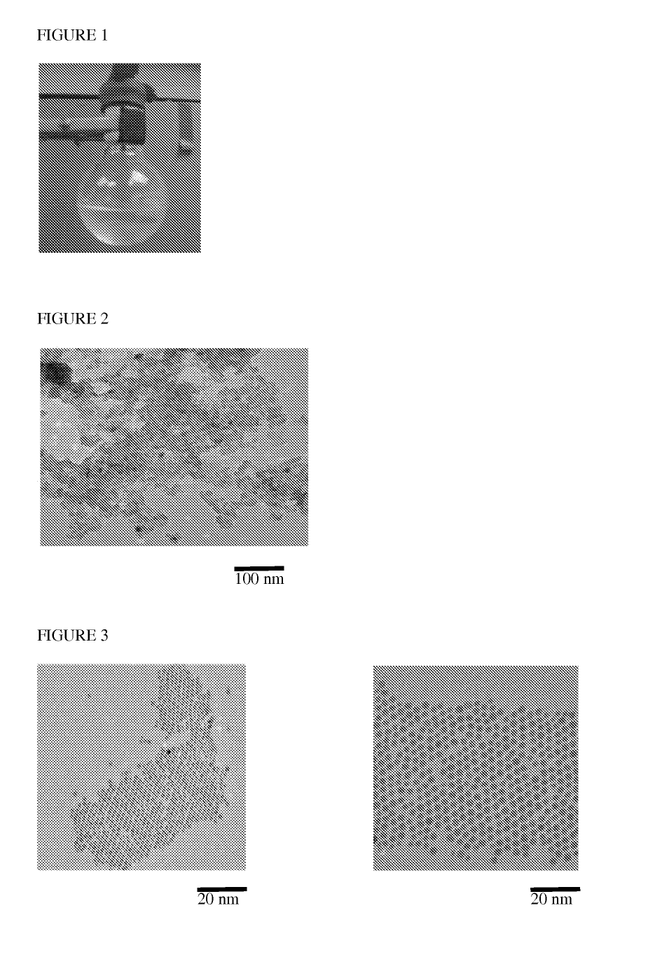

In one embodiment mixing comprises bringing the aqueous solution and the organic solution into contact in the same vessel forming a liquid biphasic system and mixing the two phases. In one embodiment 10 mL of aqueous solution and 10 mL of organic solution are mixed in the same vessel. In one embodiment a liquid biphasic system is mixed and stirred. In one embodiment the liquid biphasic system is mixed and stirred at 80° c. In one embodiment the liquid biphasic system is mixed and stirred overnight. In one embodiment the liquid biphasic system is mixed and stirred with a reflux condenser on the top. In one embodiment one result of the reaction is a toluene layer with pale yellowish color. In one embodiment one result of the reaction is a non-cloudy toluene layer. In one embodiment this result can be seen after one day of mixing and stirring at 80° c. under reflux. In one embodiment this result is different from results of experiment done with a lower ligand:PCC ratio. FIG. 1 shows one embodiment of a vessel containing the liquid biphasic system.

In one embodiment, particle collection is accomplished by separating the organic phase from the aqueous phase. In one embodiment particle collection is accomplished by separating the particles from the organic phase. In one embodiment, particle collection is conducted by evaporation of organic phase. In one embodiment collection is done by precipitation of particles from the organic phase. In one embodiment organic solvent is separated from the precipitated particles by pouring, rinsing, filtering or drying. In one embodiment particles are kept with the organic solvent for further use.

In one embodiment this invention provides a process for filling or coating a material with the nanoparticles. In one embodiment, the nanoparticles are mixed with the material. In one embodiment, the nanoparticles are spread onto the material. In one embodiment the material is a paper product. In one embodiment, the material is a food or pharmaceutical product. In one embodiment, the material is a polymer or a polymer composite. In one embodiment filling or coating a material with the nanoparticles of this invention improve the material's opacity, brightness, bulk and smoothness. In one embodiment filling or coating a material with the nanoparticles improve the material's strength or other mechanical properties. In one embodiment using the nanoparticles as a filler or a coating for a material improves the light scattering properties of the material. In one embodiment the nanoparticles of this invention give the material a high filler-retention properties.

In one embodiment this invention provides a process for synthesizing a complex nanoparticle-organic structure. The process comprises:
forming a liquid biphasic system comprising an aqueous phase and an organic phase, wherein the aqueous phase comprises calcium-containing nanoparticles and the organic phase comprises a solution of at least one organic molecule containing at least one functional end group;
mixing the liquid biphasic system to form organic molecule coated calcium-containing nanoparticles; and
linking a first organic molecule coating a nanoparticle with a second organic molecule coating another nanoparticle;
whereby the linking the two organic molecules results in the formation of an organic molecule-coated nanoparticle complex.

The processes of the invention yield nanoparticle preparations, in which any embodiment thereof as herein described may be obtained by such processes, as will be appreciated by one skilled in the art.

In some embodiments, the process as described herein, yield nanoparticles characterized by the properties described herein.

In one embodiment spacing between calcium-containing particles in the complex structure are from 1 nm to 100 nm. In one embodiment spacing between particles is 0.2 nm to 2.0 nm. In one embodiment spacing between particles is between 1 nm to 5 nm. In one embodiment, the complex structure is water permeable. In one embodiment, the complex structure serves as a filter or a membrane. In another embodiment, the complex structure serves as a coating material. In one embodiment, the complex structure serves as a column. In one embodiment, the complex structure serves as an antacid. In one embodiment the exposed end group of the coating monolayer is converted to a carboxylic acid group.

In one embodiment, the step of linking a first organic molecule coating a nanoparticle with a second organic molecule coating another nanoparticles may be desirable and is accomplished as described herein. In one embodiment organic molecules, coating two different particles, expose a carboxylic acid group to the environment. In one embodiment the environment contains diamine rod-shaped molecules wherein, the two end groups of the molecule have an amine group, and a 0.5-2.5 nm unbranched hydrocarbon chain connects the two amine groups. In one embodiment the exposed carboxylic acid groups are reacted with the diamine molecules. In one embodiment one amine group reacts with a molecule of one particle and bonds with it, and the other amine group on the same diamine molecule binds to a second carboxylic group on a second particle thus linking the two particles. In one embodiment the diamine molecule is a 1,6-diaminohexane (DAH) molecule or a O,O′-Bis(2-aminoethyl)octadecaethylene glycol (EGDA) molecule. In one embodiment the reaction of the exposed carboxylic acid groups on the particle with the divalent amine molecule forms a linkage between at least two particles. In one embodiment, multiple linkages between multiple particles form a 3D structure comprises organic molecules and inorganic particles. In one embodiment the clusters are self-standing. In one embodiment the clusters are supported by a substrate. In one embodiment the clusters fill a column.

### III. Compositions/Kits Comprising the Particles/Arrays of the Invention

In one embodiment this invention provides a composition comprising the nanoparticles of this invention. In one embodiment, such composition may be utilized for multiple applications, for example, for acid neutralization or delivery of a compound of interest. In one embodiment the composition further comprises a carrier, diluent, lubricant, flow-aid, or a mixture thereof. In one embodiment the composition is in the form of a pellet, a tablet, a capsule, a solution, a suspension, a dispersion, an emulsion, an elixir, a gel, an ointment, a cream, or a suppository. In one embodiment the composition is employed for a non-therapeutic purpose, for example, for acid neutralization of water, soil, air or in other environmental applications.

In one embodiment the composition is administered to a subject. In one embodiment the composition is in a form suitable for oral, intravenous, intraarterial, intramuscular, intracranial, intranasal, subcutaneous, parenteral, transmucosal, transdermal, or topical administration. In one embodiment the composition is a controlled release composition. In one embodiment the composition is an immediate release composition. In one embodiment the composition is a liquid dosage form. In one embodiment the composition is a solid dosage form. In one embodiment the composition further comprises an antibiotic compound, an antineoplastic compound, an immunotherapeutic agent or another drug. In one embodiment the composition further comprises a pH indicator. In one embodiment the pH indicator is a molecule. In one embodiment the pH indicator is congo red.

In one embodiment this invention provides a pharmaceutical composition comprising the nanoparticles of this invention. In one embodiment the nanoparticles composition comprises a targeted drug delivery composition. In one embodiment the nanoparticles contain a drug. In one embodiment the nanoparticles contain a cell binding function and a drug. In one embodiment the composition further comprises a carrier, diluent, lubricant, flow-aid, or a mixture thereof. In one embodiment the composition is in the form of a pellet, a tablet, a capsule, a solution, a suspension, a dispersion, an emulsion, an elixir, a gel, an ointment, a cream, or a suppository. In one embodiment the composition is in a form suitable for oral, intravenous, intraarterial, intramuscular, intracranial, intranasal, subcutaneous, parenteral, transmucosal, transdermal, or topical administration. In one embodiment the composition is a liquid dosage form. In one embodiment the composition is a solid dosage form. In one embodiment the composition further comprises an antibiotic compound, an antineoplastic compound, an immunotherapeutic agent or another drug.

In one embodiment this invention provides a tissue engineering kit comprising the nanoparticles of this invention. In one embodiment the kit comprises:
a porous complex structure of nanoparticles;
a cell source; and
   at least one, or combinations of the following components:
a composition comprising drugs or biological functions for enhancing properties such as biocompatibility, cell growth, immune system functions;
chemicals and drugs such as antibiotics, cell nutrients, oxygen, water;
cell markers, nanoparticle markers, markers for drugs or for biological functions.
reagents to remove or degrade or decompose nanoparticles structure once cell tissue is self-sustained, or during tissue growth;
a substrate, a vessel, a dish, a vial, a biological tissue, to support porous nanoparticles structure; and
a carrier, diluent, lubricant, flow-aid, or a mixture thereof.

In one embodiment this invention provides a kit for paper filling or paper coating. In one embodiment the kit includes a composition comprises nanoparticles of this invention and at least one of the following: a carrier, diluent, lubricant, solvent, flow-aid, anti-aggregation agent, adhesive, binder, dye, pigment or a mixture thereof. In one embodiment the organic coating of the nanoparticles comprises an adhesive, binder, dye, pigment, fluorescent marker, polymerization agent, complexation agent or a combination thereof. In one embodiment the kit further comprises a dispensing apparatus, tube, brush, syringe, jet, sprayer, squirt, automated dispensing system, paper running machine, press, heater, or a combination thereof.

### IV. Methods of Use of the Particles/Arrays of the Invention

In one embodiment the nanoparticle preparations of this invention may be used to pattern substrates, for example, and in some embodiments, by using an AFM tip, or in other embodiments, by spreading, evaporation, spin-coating, lithography, inkjet and other printing or patterning techniques. In one embodiment spreading the nanoparticles into a pre-patterned substrate containing wells is conducted. The particles are trapped in the wells, while in other areas on the substrates, the particles are washed away. The wells define the location of the particles. Other embodiments of this aspect of the invention include particles and methods from U.S. Pat. No. 06,989,324, fully incorporated by reference herein.

In one embodiment, particles are used to pattern substrates using an AFM tip, that is dipped in the particle solution, or that is brought into contact with a solid assembly of particles, or with single particles. The particle surface and the AFM tip surface can be coated or uncoated to promote adhesion between the AFM tip and particles. In one embodiment, once the particle or string of particles is/are attached to the tip, the tip is moved, and located over a substrate in an area where particle patterning is desired. The AFM tip is lowered until the particle at the tip touches the surface of the substrate to be patterned. Contact between the particle and the substrate is made, and the AFM tip is either lifted or moved laterally to detach the particle from the tip. The particle or particles are attached to the substrate by chemical bonds or forces. Further manipulation of the particles such as heating, coating, attaching a chemical moiety, can be conducted.

In one embodiment, the particles of this invention can be patterned onto substrate by applying a drop or a portion of a particle solution onto a substrate. The substrate is then spun, and the liquid is disposed, leaving a patterned assembly of particles on the substrate. The time and speed of such spin-coating process can define the resulting pattern. In another embodiment, instead of spin-coating, the particle solution is applied to the substrate and the solvent is left to evaporate spontaneously. In some embodiments, evaporation is at ambient conditions and in other embodiment evaporation is conducted under negative pressure and higher or lower temperatures. In some embodiment, application of the particle solution in a unidirectional stream over the substrate, defines the particle pattern that will be formed on the substrate.

In some embodiment the patterned substrate is a metal, a silicon, a silicon dioxide, indium tin oxide (ITO), polymer, paper, plastics, polydimethylsiloxane (PDMS) or other materials. In some embodiment the patterned substrate is curved. In some embodiments the Patterned substrate is biologically compatible.

In some embodiments the nanoparticles preparations of this invention may be used as scaffolds in tissue engineering. In tissue engineering, cells are often implanted or ‘seeded’ into an artificial structure capable of supporting three dimensional tissue formation. These structures, typically called scaffolds, are often critical, both ex vivo as well as in vivo, to recapitulating the in vivo milieu and allowing cells to influence their own microenvironments. Scaffolds may be useful for at least one of the following purposes:

Allowing cell attachment and migration

Delivering and retaining cells and biochemical factors

Enabling diffusion of vital cell nutrients and expressed products

Exerting certain mechanical and biological influences to modify the behavior of the cell phase;

Or combinations thereof.

Porous materials are among the numerous candidates for tissue engineering scaffolds since they can be biocompatible, resistant to biodegradation, non-toxic and can be functionalized with biomolecules.

To achieve the goal of tissue reconstruction, scaffolds must meet some specific requirements. A high porosity and an adequate pore size are necessary to facilitate cell seeding and diffusion throughout the whole structure of both cells and nutrients. Biodegradability is often an essential factor since scaffolds should preferably be absorbed by the surrounding tissues without the necessity of a surgical removal. The rate at which degradation occurs has to coincide as much as possible with the rate of tissue formation: this means that while cells are fabricating their own natural matrix structure around themselves, the scaffold is able to provide structural integrity within the body and eventually it will break down leaving the neotissue, newly formed tissue which will take over the mechanical load. Injectability is also important for clinical uses.

In some embodiment nanoparticle arrays or complex nanoparticle-organic structures of this invention can be used as scaffolds onto which cells will adhere and tissue will grow. In some embodiments CaCO₃ particles can be dissolved or degrade after cell tissue is completed. In some embodiments nanoparticle arrays can degrade slowly over a period of time necessary for the tissue build up. In some embodiments the nanoparticle can decrease in size over time, allowing controlled growth of the tissue into the newly formed spaces. In some embodiment, calcium-containing particles can degrade when exposed to a certain CO₂ concentration, a certain solution pH or other salt and chemical concentrations. In some embodiments nanoparticle arrays and structures can be used as templates for scaffold preparations known as Solvent Casting & Particulate Leaching (SCPL). The SCPL approach allows the preparation of porous structures with regular porosity. In SCPL, a polymer is dissolved into a suitable organic solvent and the solution is cast into a mold filled with porogen particles. Such porogen can be in one embodiment an array of the particles of this invention. In some embodiment such porogen is a complex nanoparticle-organic structure described in this invention. The size of the porogen particles, or the size or density of the organic linker between the particles in some embodiments will affect the size of the scaffold pores, while the polymer to porogen ratio is directly correlated to the amount of porosity of the final structure. After the polymer solution has been cast the solvent is allowed to fully evaporate, then the composite structure in the mold is immersed in a bath of a liquid suitable for dissolving the porogen. In one embodiment the liquid is a solution containing carbon dioxide, H⁺ ions or other chemicals suitable for dissolving the calcium-containing matrix. Once the porogen or complex porous structure has been fully dissolved a new polymeric porous structure is obtained. This polymeric porous structure serves as the scaffold for cell growth.

The scaffold may be useful in bone/cartilage regeneration applications, in muscle tissue engineering or in angiogenesis, or any other appropriate application for tissue growth, repair and/or regeneration. The scaffolds may be seeded with stem and/or progenitor cells to facilitate tissue reconstruction. The nanoparticles array will form in a geometry most suitable for the tissue whose generation is desired. In some embodiment, a symmetric nanoparticles array will be used. In other embodiment, the organic linker between neighboring particles will be chosen with a length that will dictate pore size between particles, and the size and morphology of the growing tissue. In some embodiments, other materials of interest may be incorporated in the scaffold, such as tissue growth promoting factors, bone morphogenic proteins or materials for bone reconstruction applications. In some embodiments, such applications may be as or comparable to that described in U.S. Pat. No. 6,592,623, fully incorporated by reference herein.

In one embodiment nanoparticles of the present invention can be used as drug delivery vehicles, wherein, the drug molecule of choice is bonded by intermolecular forces to the particle or to the particle coating. In some embodiments the particles are targeted to the location where the drug is required. In one embodiment drug action takes place while drug molecules are bounded to the particles. In one embodiment drug action takes place after drug is being released form the particle.

In one embodiment the drug molecule binds to the particle through covalent bonds and in other embodiment by polar bonds. In one embodiment the molecule is bounded to the particle by making van der Waals bonds with surface atoms of the particle. In one embodiment the drug molecule is first conjugated to a molecule that posses a functional group that can bind the particle or the coating molecules. In one embodiment the drug molecule contains at least one COOH group that can bind to the particle or to the particle coating. In one embodiment the drug molecule is the coating molecule of the particle. In one embodiment the drug molecule is one of two molecules forming the organic-layer coating of the particle. In one embodiment the drug molecule is bonded to the particle after the coating is applied, and in another embodiment the drug molecule is bonded to the particle before or during the period in which the organic coating of the particle is applied. In one embodiment the drug molecule is bonded to the particle through a linker molecule that can be cleaved using a cleaving agent. In one embodiment cleaving takes place once the particles have reached their target. In one embodiment cleavage of drug molecules is performed using a chemical. In one embodiment the cleavage is performed using electromagnetic radiation.

In one embodiment, in addition to the drug molecule, a functional group is attached to the particle such that the functional group can bind the particle to a cell or to a cell membrane. In one embodiment the function is recognized by membrane or by other cell receptors. In one embodiment the functional group contains a membrane-spanning portion.

In one embodiment a marker molecule is bounded to the molecule in addition to the drug molecule. In one embodiment the marker is a fluorescent marker. In one embodiment the marker is used to confirm the location of the particles carrying the drug, so that drug release processes can be initiated. In one embodiment the confirmation is a non-invasive confirmation. In one embodiment a core-shell particle of this invention is used. In one embodiment the core-shell particle containing a drug molecule is targeted to a location where the drug is required. In one embodiment, non-invasive opto-thermal heating of the particle releases the drug molecule from the particles. In one embodiment opto-thermal heating of the particle, induces the right conditions for the activity of the drug. In one embodiment the drug-containing particles of this invention are formulated for oral (through the mouth), nasal, aerosol (inhalation), and rectal administration. In one embodiment the particles are delivered by injection. In one embodiment the release rate of the drug molecule from the particle depends on the density of the coating. In one embodiment the release rate of the drug molecule from the particle depends on the strength of the chemical bond between the drug and the particle or the coating. In one embodiment the release rate of the drug molecule depends on natural or induced chemical or physical conditions of the environment of the particles. In one embodiment the release rate of the drug is slow. In one embodiment the release rate of the drug is over the course of days, weeks or month.

In one embodiment the particles are biodegradable. In one embodiment following or during drug release, the particles degrade or decompose. In one embodiment degradation is induced by modifying the chemical environment of the particle. In one embodiment degradation occurs following a pH change, a carbon dioxide concentration change, or salt concentration change. In one embodiment an etching agent decomposes the particles. In one embodiment particles leave the body spontaneously.

In one embodiment the nanoparticles of this inventions are used as acid neutralizers or Antacids. In one embodiment calcium-containing particles react with acid from the environment of the particles and neutralize it. In one embodiment the calcium-containing particles are calcium carbonate particles. In one embodiment the reaction of the calcium carbonate with the acid can be described by the following chemical formula: CaCO₃₍ₛ₎+2HCl_{(aq)}→CaCl_{2(aq)}+CO_{2(g)}+H₂O₍ₗ₎. In one embodiment the products of the chemical reaction are biocompatible and are spontaneously disposed. In one embodiment the rate of the acid-neutralizing reaction depends on the density of the organic monolayer coating of the particle. In one embodiment pores or defects in the organic monolayer coating give access to body fluid and permit contact between the nanoparticles and the acid protons. According to this aspect of the invention and in one embodiment the nanoparticles of this invention, when serving as antacids, follow the drug delivery methods and techniques described herein above and are being used and perform as other conventional antacids in ways that are familiar to a person skilled in the art.

In one embodiment the nanoparticles of this invention are used for optothermal targeted cancer diagnosis and therapy. In one embodiment the nanoparticles of this invention are coated with a metal layer. In one embodiment this layer and the nanoparticles are considered nanoshells. In one embodiment the nanoshells of this invention are administered to a subject. In one embodiment the nanoshells bind to tumors or cancerous cells. In one embodiment a near-infrared radiation is briefly applied over the subject's body. In one embodiment radiation scattered from the tumor-bound nanoshells, locates the tumors. In one embodiment, once located, each tumor is hit with the same radiation, at higher intensities, killing the tumor, without damaging the surrounding tissues. In other embodiments, the particles and arrays of this invention may be applied as described in U.S. Pat. No. 6,645,517, fully incorporated by reference herein.

In one embodiment, the metal shell of the nanoparticle absorbs the externally applied light energy, turning it into heat. In one embodiment the temperature of the nanoshells reaches up to 131 degrees F. In one embodiment excessive heating destroy or kill cells adjacent to the nanoshells. In one embodiment such therapy enables a single-visit diagnosis and treatment, and significantly less damage to non-cancerous tissues. In one embodiment such treatment provides high precision treatment. In one embodiment, after diagnosis and treatment, nanoshells are naturally eliminated from the body.

In one embodiment the nanoparticles of this invention function as photonic crystals. Photonic crystals are composed of periodic dielectric or metallo-dielectric structures that affect the propagation of electromagnetic waves (EM) by defining allowed and forbidden electronic energy bands. Essentially, photonic crystals contain regularly repeating internal regions of high and low dielectric constant. Photons (behaving as waves) propagate through this structure depending on their wavelength. Wavelengths of light that are allowed to travel are known as “modes”. Disallowed bands of wavelengths are called photonic band gaps. This gives rise to distinct optical phenomena such as inhibition of spontaneous emission, high-reflecting omni-directional mirrors and low-loss-waveguiding, amongst others. Since the basic physical phenomenon is based on diffraction, the periodicity of the photonic crystal structure has to be of the same length-scale as half the wavelength of the EM waves. The repeating regions of high and low dielectric constants have to be of this dimension. In one embodiment the highly monodispersed nanoparticles of this invention are arranged in a closely packed array. In one embodiment the nanoparticle array serve as photonic crystals in the soft X-ray region. In one embodiment the photonic crystals of the present invention operate as mirrors. In one embodiment the photonic crystals of this invention operate as beam splitters, filters, or as radiation protectors. In one embodiment the nanoparticles form “2D” photonic crystals and in another embodiment a “3D” photonic crystal. “2D” and “3D” are terms used in the nanoparticle and microparticle field to describe a single and multiple ordered layers of particles respectively.

In one embodiment the nanoparticles of this invention are useful in the construction of membranes or filters. In one embodiment, packing the nanoparticles of this invention into a column yields a porous material that can pass liquid and retain particles, molecules or ions. In one embodiment the chemical nature of the particle coating dictate the retention properties of the column containing the nanoparticles.

In one embodiment linking the nanoparticles with organic molecules result in a complex organic-nanoparticle structure with pore sizes that reflect the length and the density of the organic molecules linking the particles. In one embodiment the complex porous structure serves as a filter. In one embodiment a liquid solution is passed through the filter. In one embodiment a gaseous mixture is passed through the filter. In one embodiment a gas dissolved in a liquid is passed through the filter. In one embodiment the organic linkers between the particles are functionalized. In one embodiment the organic linkers are polar. In one embodiment the organic linker are non-polar. In one embodiment the organic linkers contain receptors for biological molecules. In one embodiment retention of biological molecules by the complex organic-nanoparticle complex is a method for analyzing a biological sample.

In one embodiment the nanoparticles of this invention are contained in paper products. In one embodiment the nanoparticles of this invention are used as paper fillers. In one embodiment the nanoparticles of this invention serve as paper coaters. In one embodiment filling or coating a paper with the nanoparticles of this invention enhance the brightness of the paper. In one embodiment filling or coating a paper with the particles of this invention enhance the bulk or the opacity of a paper. In one embodiment, filling or coating a paper with the nanoparticles of this invention controls the weight, the surface gloss, smoothness or ink absorbency of the paper. In one embodiment and based on the nanoparticle surface, coating a paper with the nanoparticle protects it against ultraviolet radiation. In one embodiment filling a paper with the nanoparticles improves paper properties such as good opacity, light scattering ability, brightness, bulk, smoothness, gloss and retention properties. Improved strength properties, good dewatering properties that can lead to improvement in drainage and machine runnability. The nanoparticles of this invention when contained in or on a paper can have high oil absorption characteristics for benefits in paint and coatings.

In one embodiment the organic monolayer coating can be chosen such that the organic monolayer will affect the paper characteristics. In one embodiment the organic monolayer is hydrophobic. In one embodiment the organic monolayer is converted to a hydrophilic monolayer. In one embodiment oxidation of the monolayer convert the exposed end groups of the monolayer to COOH groups which are hydrophilic. In one embodiment the organic monolayer contain dyes that affect the color of the paper. In one embodiment the surface properties of the nanoparticles (bare or coated) can be modified to give the desired paper properties described herein above in chemical ways that are known to a person skilled in the art. Other embodiments include particles and methods from U.S. Pat. No. 04,943,324, fully incorporated by reference herein.

In one embodiment the nanoparticles of this invention serve as a component of ink or paint. In one embodiment the nanoparticles of this invention are incorporated in electronic inks. In one embodiment, chemical properties of the nanoparticles of this invention can be modified to enhance ink or paint properties such as absorbancy in media, durability, solubility, color gamut, viscosity and electronic charge.

In one embodiment the polymeric-shell particles of this invention are used as additives to paper products, plastics, ink, paint, drug formulations, cosmetics preparations, textiles, packaging materials, adhesive tapes, strong composite materials for aerospace vehicles or for protective wear. In one embodiment fluorescent polymeric-shell particles are used for imaging. In one embodiment controlled release of drugs or pesticides is achieved by polymeric encapsulation. In one embodiment the polymer encapsulated particles are used in textiles.

In one embodiment, the term “a” or “one” or “an” refers to at least one. In one embodiment the phrase “two or more” may be of any denomination, which will suit a particular purpose. In one embodiment, “about” may comprise a deviance from the indicated term of +1%, or in some embodiments, −1%, or in some embodiments, ±2.5%, or in some embodiments, ±5% or in some embodiments, ±7.5%, or in some embodiments, ±10%, or in some embodiments, ±15%, or in some embodiments, ±20%, or in some embodiments, ±25%.

In one embodiment the nanoparticle serves as a drug. According to this aspect of the invention and in one embodiment, the nanoparticle serves an antacid. In one embodiment the calcium containing antacid material is encapsulated in a polymeric matrix. In one embodiment the calcium-containing antacid is coated with an organic monolayer. In one embodiment the antacid is coated by multilayers of organic molecules. In one embodiment the ordered multilayers are formed step-by-step, one layer at a time. In one embodiment such step-by-step construction is performed by reacting the surface functional group of a bounded molecule with the first functional end group of a bifunctional molecule introduced from the environment. In one embodiment after each binding step, the second functional end group of a newly bound molecule is exposed and can bind an additional molecule. In one embodiment this process is repeated. In one embodiment the antacid coating is porous. In one embodiment the antacid coating is permeable. In one embodiment the antacid coating is biocompatible or non-toxic. In one embodiment the antacid coating is biodegradable. In one embodiment the antacid coating is colored.

In some embodiments this invention may be used for enforcing a material.

In one embodiment the nanoparticle serves as a filling material. In one embodiment the nanoparticle serves as a paper filling material. In one embodiment the high monodispersion level of the particles gives the paper unique properties. In one embodiment the small size distribution of the particles improves the paper brightness, opacity and bulk. In one embodiment the coating of the particle improves adhesion of particles to other paper components or pulp. In one embodiment the paper is a printing or a writing paper. In one embodiment the nanoparticle serves as a coating material. In one embodiment the nanoparticle serves as a paper coating material. In one embodiment the particles serving as a coating are closely-packed. In one embodiment the organic coating of the closely-packed particles contain a dye. In one embodiment coating a paper with dyed-particles of this invention results in a colored paper. In one embodiment the paper is a printing or a writing paper. In one embodiment the nanoparticle is used for coating a polymer, a silicon substrate, glass, metal or inorganic transparent material.

In one embodiment the small size distribution and the spherical geometry of the particles enable the particles to assemble in a closed-packed, periodically ordered array. In one embodiment a single periodic layer is formed. In one embodiment such single layer is called a 2D structure as known to a person skilled in the art. In one embodiment the 2D structure of the nanoparticles is formed on a substrate. In one embodiment a 2D structure is formed on a glass, a silicon, a metal, TiO₂, a polymeric structure or a TEM grid. In one embodiment the nanoparticles form multiple ordered arrays known as “3D” structures. In one embodiment such 2D and 3D periodic arrays attenuate light. In one embodiment such arrays reflect or diffract or transmit components of light. In one embodiment such 2D and 3D structures are used to form mirrors, or photonic devices. In one embodiment the photonic devices reflect or diffract or transmit a restricted range of wavelengths. In one embodiment the wavelength diffracted or transmitted or reflected are in the soft-X-ray region.

### EXAMPLES

### Example 1

### Synthesis of Highly Monodispersed, Organic Monolayer Coated, Precipitated Calcium Carbonate (PCC) Nanoparticles

A synthesis of highly monodispersed, organic monolayer coated, precipitated calcium carbonate (PCC) nanoparticles was carried out by reacting a 1:10 PCC:ligand mixture. The mixture contained an aqueous phase and an organic phase. The aqueous phase contained the PCC particles and the organic phase contained the organic molecules (the ligands).

The aqueous phase containing the PCC particles was prepared as follows:

847 mg of the PCC particles, PCC particles having a specific surface area of about 80 m²/g and are made according to example 2 of U.S. Pat. No. 5,643,631, the disclosure of which is incorporated herein by reference, were obtained. 11.8% by weight aqueous PCC solution which contains 100 mg, 1 mmol of CaCO₃ nanoparticles was diluted to 10 mL final volume with distilled water. FIG. 2 is a TEM image showing the PCC particles used.

Organic phase containing the ligand mixture was prepared as follows: A ligand mixture of 9-decenoic acid(DA) and 4-phenyl butyric acid(PBA) (1:1) mole ratio was prepared. The mixture contains: 850 mg, 5 mmol of 9-decenoic acid and 820 mg, 5 mmol of 4-phenyl butyric acid. Ligand mixture was dissolved in 10 mL of toluene.

Total mole ratio of PCC:Ligand is 1 mmol: 10 mmol.

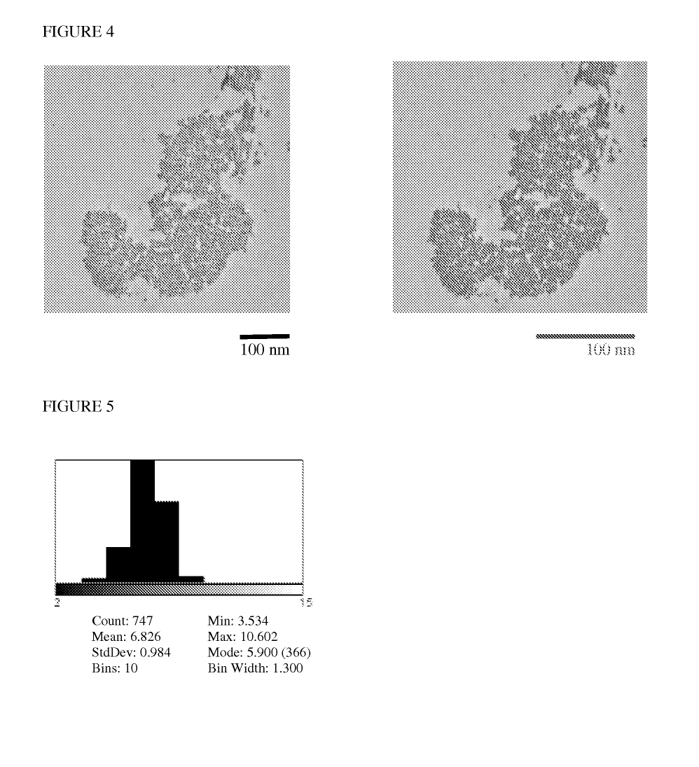

Aqueous phase and organic phase were mixed in a vessel (FIG. 1) and stirred at 80° c. overnight with reflux condenser on the top. The next day toluene layer had pale yellowish color and it was not cloudy looking unlike previous cases with lower ratio of ligand:PCC ratio. Sample was taken from the toluene layer for TEM microscopy, and it contained uniform spherical particles as shown in FIG. 3. The resulting nanoparticles were highly monodispersed and they were forming two dimensional arrays as shown in FIG. 3. Size distribution analysis on these particles was performed using image J program analysis. The nanoparticles population analyzed is shown in FIG. 4. Size distribution plot for a population of 747 particles is shown in FIG. 5. The mean diameter was found to be 6.8 nm and the mean standard deviation was 1.0 nm. The smallest particle found had a diameter of 3.5 nm and the largest particle found had a diameter of 10.6 nm. TEM diffraction analysis has been performed on a large two dimensional nanoparticle array and it has been confirmed that particle are composed of calcium carbonate.

### Example 2

### Using Organic Molecules to Extract Calcium Carbonate Particles from Hard Water

Calcium carbonate is the main constituent of hard water. Hard water presents a problem when used for drinking and when used in house hold and commercial appliances and systems where water at high temperatures are used. Calcium carbonate can accumulate and block the tubes, valves, inlets of water boilers, cooling systems, laundry machines, soft drinks plants and other machinery. In one embodiment of the current invention, a way to soften hard water is proposed. Hard water in which CaCO₃ particles are small will be placed in contact with an organic phase. The organic phase will consist of an organic solvent and two types of organic molecules with a carboxylic head group. The aqueous and organic phases will be mixed and stirred, and the organic molecules will adhere to the calcium carbonate particles through the carboxylic head group. This will form a uniform organic monolayer around the particles. The hydrophobic, organic alkyl chains of the molecules are exposed to the solvent. This will result in a non-polar surface which will render the particles soluble in the organic solvent and will remove it from the water phase. A simple separation of the two phases will follow, resulting in soft, pure water as one phase and in an organic phase containing coated calcium carbonate particles. The organic layer can then be stripped by oxidation, and the particles can precipitate from the organic solvent into a solid phase, leaving the solvent clear for subsequent use.

### Example 3

### Enhancing Brightness and Opacity of Printing Paper

Precipitated calcium carbonate (PCC) is well known for high performance in paper coatings. Scientists have correlated increases in coating structure to the simple concept of maximizing the number of particles of the “right” particle size and shape that can scatter light effectively. In one embodiment, the present invention provides precise control over the PCC crystal structure, average particle size and particle size distribution.

In selecting pigments for a coating formulation, control over all three pigment properties (particle shape, size and distribution) determines the final coating structure, its optical efficiency, and the resulting coated paper performance. Specifically, particle shape can improve coating structure through physical hindrance while average particle size and particle size distribution can provide coating structure through controlled consolidation. It can provide improved packing of particles which results in enhanced surface properties. The average particle size and narrow size distribution can also provide optical efficiency. In this embodiment of the present invention monodispersed PCC nanoparticles will be formed with preferred geometry for special paper coating application. Varying the experimental conditions such as temperature, type of organic solvent, type of organic molecules, stirring time etc. various geometries and size ranges can be obtained. The organic monolayer coating of the nanoparticles can result in an extra level of performance of the coated paper in terms of brightness, opacity, smoothness, ink adhesion, sheet gloss, print gloss and surface strength.

### Example 4

### Controlled Release of Calcium Carbonate for Acid-Neutralizing

Acid neutralizers tablets (antacids) and acid neutralizing processes in natural water reservoirs and in industrial water are important for the relief of acid associated pain for people with ulcers or with gastroesophageal reflux disease, or acid indigestion. In water reservoirs, acidic conditions caused by acid rain or acidic pollutants can destroy the ecological system and can pose serious environmental effects. In industrial processes acidic water can interfere with the process and with the product yield. Calcium carbonate is often used as an antacid. The reaction of calcium carbonate with acid is described by the following chemical formula: CaCO₃+2HCl→CaCl₂+CO₂+H₂O. The products of this reaction are the soluble, harmless calcium chloride, gaseous carbon dioxide which is easily released from the system and water.

In one embodiment of the present invention, monodispersed nanoparticles coated by organic molecules will be used as antacids. The particles can be introduced to the acidic acid solution as a solid and mixed with it. If higher solubility of particles in an aqueous phase is needed, the organic monolayer covering the particles can be modified by e.g. oxidation resulting in a polar group at the exposed surface of the particles. The monolayer structure on the calcium carbonate particles can be designed to have small holes and or pores so that controlled and slow contact of the calcium carbonate and the acidic solution is obtained. This will cause slow release of the calcium carbonate available for reaction. This slow release is important in cases where sudden gaseous CO₂ production can interfere with process conditions and when the acidic phase is mobile and the level of acidity should be decreased gradually. The head groups of the organic monolayer can be further modified with a linker that will chemically link the nanoparticles to each other. This linkage will form a porous material with exposed calcium carbonate surface areas (in areas of monolayer defects). Such material can be used as a filter or a membrane or as part of a device that will act as a controlled antacid. Variation in particle size, geometry, density and in monolayer coverage and properties will modify acid-neutralizing properties of device.

What is claimed is:

1. A process for preparing monodispersed organic monolayer-coated calcium-containing nanoparticles, said method comprising the steps of:
a. forming a liquid biphasic system comprising an aqueous phase and an organic phase, wherein said aqueous phase comprises calcium-containing nanoparticles and said organic phase comprises a solution of at least one organic molecule containing at least one functional end group;
b. mixing said liquid biphasic system; and
c. collecting nanoparticles from the organic phase of said liquid biphasic system;
whereby said at least one organic molecule containing a functional end group interacts with said nanoparticles, such that said at least one organic molecule coats said nanoparticles.

2. The process of claim 1, wherein nanoparticles collected in step (c) are closely packed.

3. The process of claim 1, wherein said mixing is conducted at 80° c. for at least 12 hours.

4. The process of claim 1, wherein said mixing is conducted under reflux.

5. The process of claim 1, wherein said at least one functional end group is a carboxylic group, an amine or a combination thereof.

6. The process of claim 1, wherein said collecting is accomplished by separating of said organic phase from said aqueous phase.

7. The process of claim 1, wherein said collecting is conducted by evaporation of said organic phase.

8. The process of claim 1, wherein said calcium-containing nanoparticle is a calcium carbonate nanoparticle.

9. The process of claim 1, wherein said nanoparticles have a diameter ranging from between 1 nm-100 nm.

10. The process of claim 1, wherein the geometry of said nanoparticles collected in step (c) is more spherical in shape than said nanoparticles in step (a).

11. The process of claim 1, wherein the size distribution of said nanoparticles collected in step (c) ranges between 5%-20% of the mean particle size.

12. The process of claim 1, wherein said nanoparticles form an ordered array.

13. The process of claim 1, wherein said solution comprises two or more organic molecules.

14. The process of claim 14, wherein said two or more organic molecules comprises at least one hydrophilic molecule and at least one hydrophobic molecule.

15. The process of claim 14, wherein said two or more organic molecules comprise 9-decenoic acid and 4-phenyl butyric acid.

16. The process of claim 1, wherein said step (c) results in nanoparticles having a coating comprising between 60% to 98% of their surface area.

17. The process of claim 1, wherein said step (c) results in nanoparticles having a coating comprising between 10% to 40% of their surface area.

18. The process of claim 1, wherein said nanoparticles are thermally stable.

19. The process of claim 1, wherein said nanoparticles are nontoxic for human consumption.

20. The process of claim 20, wherein said nanoparticles are administered as an antacid.

21. The process of claim 1, wherein a material of interest is adsorbed or deposited on said organic molecule coating said nanoparticles.

22. The process of claim 21, wherein said material of interest comprises a metal, semiconductor or targeting moiety.

23. The process of claim 1, further comprising the step of applying a metal coating to said particles.

24. The process of claim 23, wherein said applying comprises introducing a metal ion precursor to an organic phase comprising said coated nanoparticles, and providing conditions such that metal precipitation occurs.

25. The process of claim 1, further comprising the step of applying a second organic molecule coating to said particles.

26. The process of claim 25, wherein said applying comprises introducing a functionalized organic molecule to an organic phase comprising said nanoparticles and providing conditions such that said second organic molecule bonds or adheres to said calcium-containing nanoparticles.

27. The process of claim 24, wherein said organic molecule is a polymer or a biological molecule or a biological function.

28. The process of claim 1, further comprising the step of precipitating said nanoparticles and thereby forming a desired ordered structure.

29. The process of claim 28, wherein said desired ordered structure is in the form of a lattice comprising multiple ordered layers of said nanoparticles.

30. The process of claim 1 further comprising applying said nanoparticles obtained in (c) as a coating to a material, or incorporating said nanoparticles within a product.

31. The process of claim 30, wherein said nanoparticles are applied as a coating to a paper product.

32. The process of claim 30 wherein said nanoparticles comprise a filler material.

33. The process of claim 32, wherein said filler material is utilized in the preparation of a food or pharmaceutical product.

34. The process of claim 32, wherein said filler material is utilized to enhance the mechanical or optical properties of a polymer or a polymer composite.

35. A device, apparatus or apparel comprising nanoparticles prepared according to the process of claim 1.

36. A paper or a drug comprising the nanoparticles prepared according to the process of claim 1.

37. A process of synthesizing an organic molecule-coated nanoparticle complex, said process comprising:
a. forming a liquid biphasic system comprising an aqueous phase and an organic phase, wherein said aqueous phase comprises calcium-containing nanoparticles and said organic phase comprises a solution of at least one organic molecule containing at least one functional end group;
b. mixing said liquid biphasic system to form organic molecule coated calcium-containing nanoparticles; and
c. linking a first organic molecule coating a nanoparticle with a second organic molecule coating another nanoparticle;
whereby said linking in step (c) results in the formation of an organic molecule-coated nanoparticle complex.

38. The process of claim 37, wherein spacing between calcium-containing nanoparticles in said complex is about 1 nm-100 nm.

39. The process of claim 37, wherein said complex is water permeable.

40. The process of claim 37, wherein said complex serves as a filter or a membrane.

41. The process of claim 37, wherein said complex serves as a coating or a filling material.

42. The process of claim 37, wherein said complex serves as an antacid.

43. Highly monodispersed, organic-monolayer coated, calcium-containing nanoparticles

44. The nanoparticles of claim 43, wherein said calcium-containing material is calcium carbonate.

45. The nanoparticles of claim 43, wherein said nanoparticles have a spherical geometry.

46. The nanoparticles of claim 43, wherein said nanoparticles have a diameter ranging from between about 1 nm-100 nm.

47. The nanoparticles of claim 43, wherein said nanoparticles have a diameter ranging from between about 5 nm-10 nm.

48. The nanoparticles of claim 43, wherein said nanoparticles coating thickness ranges from between about 0.5 nm-3.0 nm.

49. The nanoparticles of claim 43, wherein said nanoparticles are closely-packed.

50. The nanoparticles of claim 43, wherein the size distribution of a particle population represents a normal distribution.
